(19) United States
(12) Patent Application Publication
(10) Pub. No.: US 2005/0042302 A1
(43) Pub. Date: Feb. 24, 2005

(54) Mixture of base-containing micronutrient substances

(76) Inventors: Norbert Fuchs, Mariapfarr (AT); Peter Kossler, Mariapfarr (AT)

Correspondence Address: FULBRIGHT & JAWORSKI L.L.P., 600 CONGRESS AVE., SUITE 2400, AUSTIN, TX, 78701, US

(21) Appl. No.: 10/490,926

(22) PCT Filed: Sep. 25, 2002
(86) PCT No.: PCT/AT02/00279

(30) Foreign Application Priority Data
Sep. 27, 2001 (AT) 1534/2001

(51) Int. Cl. A61K 31/525; A61K 31/51; A61K 33/00; A61K 33/04; A61K 33/24; A61K 33/26

(52) U.S. Cl. 424/622; 424/643; 424/656; 424/717; 424/702; 424/646; 514/574; 514/474; 514/251; 514/276; 514/350; 514/356

(57) ABSTRACT

There is described the use of organic acid salts to accelerate lactate degradation.

The invention relates to base-containing micronutrient mixtures

In addition to perfect training and the right mental attitude, selective sports nutrition is considered one of the pillars to provide optimum physical fitness. Yet, it is exactly that selective sports nutrition which has constantly been under-estimated as one of the prerequisites to provide optimum efficiency.

On account of the evidenced relationship between micronutrient supply deficits and efficiency drops, the supply of an athlete with micronutrients and the assessment of the metabolism of individual micronutrients during acute and chronic excessive physical loads have gained increasing importance.

By far the major portion of the energy required during physical labour, in particular during extended workout periods, is provided by the aerobic oxidation of nutrients. The second option to make energy available, i.e. anaerobic oxidation, is followed if the instantaneous energy demand cannot be met by aerobic oxidation. The energy supply capacity by aerobic oxidation is limited, above all, by:
the oxygen availability to the target cell,
the capacity of various enzyme systems catalyzing the individual reactions during oxidation,
the intercellular and humoral buffering capacity values.

The key element of anaerobic energy supply (anaerobic glycolysis) is the formation of lactate. Up to a certain load intensity, the degradation of lactate in the body occurs faster than its production. If the lactate produced can only just be degraded by the body, this is referred to as “anaerobic threshold”. If, however, this threshold is exceeded, the lactate produced can no longer be eliminated quickly enough by the body. Hence follows a significant increase in muscle and blood lactate counts. The lactic acid accumulating in a muscle cell changes the intracellular pH and restricts enzyme activities. Although the major portion of the H⁺-ions formed are buffered by bicarbonate, and the lactic acid is relatively quickly released above all into blood, the muscles tire out forcing the organism to throttle or completely stop its activity.

At rest, the lactate value amounts to approximately 0.5-1 mmol/l blood. Under maximum physical loads, this value can rise up to 20-30 mmol/l. On the anaerobic threshold (AT), the lactate value is about 5 mmol/l blood.

After exertion, the elimination of the accumulated lactate occurs partially within the cell by reconstruction to glycogen or further processing with an energy recovery in the aerobic metabolism. From blood, lactic acid is eliminated by oxidative combustion via the heart muscle and its uptake by the liver, kidneys and unstrained muscles. The rate of elimination from blood is 0.5 mmol/min at lactate concentrations of >5 mmol/l. In the context of lactate production, also the buffering capacities of blood and muscles are of relevance. The lactate transgressing into blood is predominantly buffered by the bicarbonate buffer system.

Isotonic or hypotonic sports drinks that have so far been available in the prior art exclusively aim to supply water, carbohydrates or electrolytes. The selective influence of the anaerobic threshold performance and buffer capacities in order to improve the aerobic energy potential, however, cannot be reached by such means.

It is, therefore, the object of the present invention to improve the aerobic energy potential and provide a composition by which such an improved aerobic energy potential will be obtained.

According to the invention, this object is achieved by the use of organic acid salts to accelerate lactate degradation. Furthermore, the invention contemplates the use of such organic acid salts for improving endurance and efficiency. According to the invention, it has also been shown that organic acid salts can be used to increase the anaerobic threshold performance. According to the invention, organic acid salts are also used to prevent or slow down symptoms of fatigue. Such salts of organic acids are, thus, particularly well suited for use as bases for sports drinks.

According to the-invention, preferred organic acid salts are salts of organic acids having a C₁-C₆ base body such as, in particular, carbonates, hydrogen carbonates, citrates, hydrogen citrates, acetates, gluconates or tartrates and, in particular, carbonates or hydrogen carbonates and citrates, hydrogen citrates or salts of other C₂-C₆ acids. The salts according to the invention can be used alone or as combinations of two or more of such salts.

In a preferred manner, Na, K, NH₄, Ca or Mg salts of organic acids can be used.

According to another aspect, the present invention relates to a base-containing micronutrient mixture containing organic acid salts, B-complex vitamins, vitamin C, iron, chromium, selenium, zinc, manganese and copper, dissolved, said micronutrient mixture having an osmotic pressure of 760 kPa or less, preferably 750 kPa or less, in an aqueous solution. The salts of organic acids can be selected from a single salt species or mixtures of different salts. Preferably, the composition according to the invention contains one or more of the salts set out above as preferred salts. This composition according to the invention is a special base suitable for the uses according to the invention such as, e.g., sports drinks, since it is able to meet all of the objects of the present invention in a particularly advantageous manner.

It has turned out in a surprising manner that such uses according to the invention and, in particular, the preparation according to the invention are able to markedly improve the aerobic energy potential by
increasing the oxygen availability to the target cells through selective physical training,
optimizing the capacity of redox enzymes through selective micronutrient intake, and
enhancing the intercellular and humoral buffering capacities through selective alkaline electrolyte supply.

All of these effects can now be reached by the use according to the invention on account of the selectively chosen ingredients.

It has been shown that, in addition to organic acid salts and, in particular hydrogen carbonates, also vitamins and trace elements are advantageous as enzyme activators to neutralize the lactate accumulating during physical exercising etc., on account of the anaerobic energy supply and the thus caused rise in the individualized anaerobic threshold performance (IAT, measured in watts). The generation of energy through anaerobic oxidation is an alternative for the cell to provide the increased quantity of energy required, even if the enzyme systems of the respiratory chain have reached their maximum transformation rate or capacity.

In the end, it is not solely the oxygen available in the mitochondria which is to be regarded as a factor limiting the aerobic energy supply, but the depleted capacity of the enzyme systems (caused by vitamin and trace element deficits) further processing the pyruvate to aerobically provide energy, which forces the cell to meet the elevated energy demand via the anaerobic oxidation option. The result is an increased lactate production.

The vitamins and trace elements admixed to the preparation according to the invention increase the capacity of enzyme systems and improve the oxygen availability in the cells. Of particular relevance in this context are the trace elements iron, chromium, selenium, zinc, manganese and copper as well as the B-complex vitamins and vitamin C.

Trace elements are components of enzymes or other active substances. Thus, they intervene in various metabolic sectors in a regulatory manner. Iron, zinc, chromium and selenium are of special importance from a sports-medical aspect.

Being, in particular, a component of oxygen-transmitting active groups (haemoglobin, myoglobin, cytochromes), iron fulfils a variety of metabolic functions. Sufficient iron supply is particularly important to athletes because of the increased oxygen transport demand in blood and the higher blood quantity formed by the organism. If the iron supply is in deficit, an insufficient amount of erythrocytes will be formed and the blood's capacity to transport oxygen will be restricted. The consequences of an insufficient oxygen supply due to a non-optimum iron supply include exhaustion, fatigue, lack of concentration, premature hyperacidity of the muscles, insomnia and circulatory disorders. The combination of iron and vitamin C in the preparation enhances iron absorption (vitamin C being considered an adsorption promoter).

Zinc is an essential trace element and a component and activator of some 100 enzymes of the intermediary metabolism. Being a carboanhydrase component, zinc supports the reabsorption of acid-binding bicarbonate, thus favourably contributing to the regulation of the acid-base balance.

Chromium is an essential element of the carbohydrate metabolism. B-complex vitamins are essential co-factors in the protein, fat and carbohydrate metabolisms;

While isotonic or hypotonic drinks according to the prior art exclusively serve rehydration, the preparation according to the invention, and the use according to the invention, ensure a retarded lactate surge and hence an improved IAT in addition to that rehydration effect. Since lactate under physical stress constitutes the primary performance-limiting factor, an improved IAT in practice means an optimized aerobic efficiency not only to leisure and serious athletes but also to non-athletes, due to the optimization of the redox-enzyme system (by micronutrients) and the enhancement of the buffer capacities by alkaline electrolytes.

By contrast, rehydration drinks, i.e. isotonic and hypotonic thirst quenchers according to the prior art, are above all concerned with the effects of carbohydrate and electrolyte additions. The preparation according to the invention, however, meets the demands for an optimum sports drink in the sense of an increased cellular enzyme activity and consequently improved exploitation of the aerobic metabolism, which results in a retarded and reduced lactate surge.

The micronutrient mixture according to the invention in an aqueous solution preferably has an osmotic pressure of 700 kPa or less, preferably 650 kPa or less, and lies thus clearly below 799.5 kPa, the value for a physiologic saline solution, or 763.0 kPa, the value for blood at 37° C.

The micronutrient mixture according to the invention in an aqueous solution preferably has a pH of 7.5 or more, more preferably 8.0 or more and, in particular, 8.5 or more. In a preferred manner, the micronutrient mixture according to the invention is base-binding in vivo. This can be ensured not only by the basic hydrogen carbonate and carbonate electrolytes, but also by other organic acid salts (e.g., gluconates, citrates, hydrogen citrates . . . ).

In addition to the ingredient components provided according to the invention, also other ingredients can be provided in the present preparation, in particular vitamins and trace elements (as enzyme activators). Therefore, the composition according to the invention preferably further contains vitamin E, provitamin A, molybdenum, magnesium, chloride, sodium, calcium, potassium, phosphate or mixtures of these substances.

As already pointed out, the preparation according to the invention differs from conventional isotonic drinks available on the market, which are based primarily on the supply of water, electrolytes and carbohydrates. By contrast, the preparation according to the invention preferably has a low carbohydrate content of less than 30%, particularly less than 20%, based on the total weight of the dry composition.

Furthermore, the present composition is substantially free of fats, i.e. its fat content is below 1%, preferably below 0.5%. Likewise preferred is a composition which is substantially free of proteins, having a protein content of, for instance, below 2% and, in particularly, below 1%.

Alternatively, a variant of the composition according to the invention may, however, also comprise highly unsaturated fatty acids (derived, e.g., from vegetable oils left to nature).

B-complex vitamins are preferably selected from vitamins B1, B2, B6, B12, biotin, folic acid, pantothenic acid, niacin and mixtures thereof.

The ingredients of the present invention can preferably be provided in forms that enable easy intake/absorption by the body. Especially preferred are those salt forms of the ingredients according to the invention, which have hitherto proven their excellent physiologic absorption capacity. Thus, anionic/cationic salt forms, ester forms, hydrochlorides or hydrate salts or similar derivatives may each be preferred as a function of the individual component.

According to preferred embodiments of the composition according to the invention as regards the relative quantitative ratios of the individual components, it comprises independently salts of organic acids in amounts of from 0.2 to 20%, preferably 1 to 10%, in particular 2 to 7%; B-complex vitamins in amounts of from 0.0001 to 2%, preferably 0.001 to 1%, in particular 0.01 to 0.5%; vitamin C in an amount of from 0.001 to 5%, preferably 0.01 to 2%, in particular 0.1 to 1%; iron in an amount of from 0.0001 to 0.5%, preferably 0.001 to 0.2%, in particular 0.01 to 0.1%; chromium in an amount of from 0.000001 to 0.01%, preferably 0.00001 to 0.001%, in particular 0.0001 to 0.001%; selenium in an amount of from 0.000001 to 0.01%, preferably 0.00001 to 0.001%, in particular 0.0001 to 0.001%; zinc in an amount of from 0.0001 to 0.5%, preferably 0.001 to 0.2%, in particular 0.01 to 0.1%; manganese in an amount of from 0.00001 to 0.1%, preferably 0.0001 to 0.01%, in particular 0.001 to 0.01%; and copper in an amount of from 0.00001 to 0.1%, preferably 0.0001 to 0.01%, in particular 0.001 to 0.01%; each based on the total weight of the dry composition.

As already mentioned, the values indicated for the individual ingredients are to be considered independently. Yet, a preferred embodiment of the composition according to the invention relates to a composition in which all of the relative quantities indicated above are contained in the percentages indicated.

In addition, the composition according to the invention preferably comprises carrot and other vegetable extracts, orange and other fruit powders, citric acid and other organic acids, beta-carotene, anthocyans and other colouring agents as well as aspartames and other sweetening agents.

The composition according to the invention is preferably administered in an aqueous solution and in this form can, therefore, be readily made available as a sports drink.

It is preferably made available in a dried, storage-stable form for that period of time over which it is to be stored and sold. Its liquid form is preferred for immediate use, e.g. as a sports drink, whereas the dry composition is preferred in the context of production and marketing processes.

The composition according to the invention preferably is present in dose form, or sold in dose form, preferably in a daily dose form or single dose form to be consumed, for instance, two to five times and, in particular, three times a day. These dose forms can be provided already in liquid forms, whereas concentrates are preferably provided in dried, storage-stable forms.

The composition according to the invention preferably comprises cyanocobalamine, sodium selenite, sodium molybdate, chromium-III-chloride hexahydrate, riboflavin, aneurin-HCl, pyridoxine-HCl, calcium pantothenate, dl-alpha-tocopherol, copper gluconate, manganese gluconate, zinc gluconate, iron gluconate, sodium ascorbate, or mixtures thereof. These special forms of the components according to the invention have turned out to be particularly well suited for an efficient intake of the composition according to the invention.

Furthermore, the composition according to the invention in a preferred manner comprises citric acid and other acidifying agents, orange fruit powder and other fruit extracts and flavours, carrot extract and other fruit and vegetable extracts, calcium glycerophosphate, magnesium glycerophosphate, sodium chloride, sweetening agents, in particular aspartame, and others or mixtures of these components.

As already indicated, the composition according to the invention preferably is administered as an aqueous solution to be consumed at a concentration of from 0.5 to 200 g, in particular 2 to 70 g, in particular 5 to 20 g, each based on the dry weight per 250 ml water.

In a preferred manner, the composition according to the invention comprises sodium, ammonium, potassium hydrogen carbonates or mixtures thereof, and also carbonates, gluconates, citrates, mono- and dihydrogen citrates, tartrates, and salts of other organic acids, or mixtures thereof.

Selenium is preferably added to the present composition in the selenite or selenate form. The metals are preferably provided in gluconate form or in other well tolerated and readily absorbable forms.

In a preferred manner, the composition according to the invention comprises sodium, potassium, calcium, magnesium carbonates and bicarbonates; sodium, potassium, calcium, magnesium salts of organic acids, in particular citrates or tartrates; or mixtures of these components.

As already pointed out, it has become feasible by the present invention to increase the oxygen availability to the target cell, to optimize the capacity of the redox enzymes, and to enhance the intercellular and humoral buffer capacities.

According to a further aspect, the present invention, therefore, relates to the use of the composition according to the invention to accelerate lactate degradation in order to enhance endurance and efficiency, increase the anaerobic threshold performance, and prevent or retard symptoms of fatigue.

These uses are particularly beneficial as bases for performance-increasing and -prolonging drinks. A preferred application, therefore, is the use of the composition according to the invention in the context of sports activities and, in particular, exercises and regeneration.

The invention will be explained in more detail by way of the following examples, to which it is, of course, not limited.

### EXAMPLES

Methodology:

Design of study: monocentric, placebo-controlled, double-blind

Center of study: Institut SportMed, Institut für sportwissen-schaftliche Leistungsdiagnostik-Trainingssteuerung-Forschung, Vienna

Participants: 40 persons (26 males, 14 females, aged between 20 and 40 years); 20 verums, 20 placebos.

Duration of study: 10 weeks

Test substance: The participants in the study consumed a preparation of the following composition three times a day (about 9.3 g each, dissolved in 0.25 1 water, half an hour before each meal):

Ingredients: Acidifying agents, citric acid, calcium glycerophosphate, orange fruit powder, potassium hydrogen carbonate, carrot extract, magnesium glycerophosphate, sodium chloride, sodium hydrogen carbonate, sodium ascorbate, beta-carotene, iron gluconate, zinc gluconate, sweetening agent aspartame, manganese gluconate, niacin, copper gluconate, vitamin E, pantothenic acid, vitamin B6, vitamin B1, vitamin B2, chromium-III-chloride hexahydrate, sodium molybdate,. sodium selenite, folic acid, biotin, vitamin B12.

TABLE 1

| Daily dose (corresponding to 27.9 g): | | | | | |
|---|---|---|---|---|---|
| Vitamin C | 150.00 | mg | Sodium | 319.50 | mg |
| Niacin | 18.00 | mg | Chloride | 272.46 | mg |
| Vitamin E | 9.99 | mg | Magnesium | 249.99 | mg |
| Pantothenic acid | 6.00 | mg | Iron | 15.00 | mg |
| Vitamin B6 | 2.01 | mg | Zinc | 15.00 | mg |
| beta-carotene | 1.62 | mg | Manganese | 5.01 | mg |
| Vitamin B2 | 1.59 | mg | Copper | 2.01 | mg |
| Vitamin B1 | 1.41 | mg | Molybdenum | 200.00 | mcg |
| Folic acid | 200.00 | mcg | Chromium | 200.00 | mcg |
| Biotin | 50.00 | mcg | Selenium | 100.00 | mcg |
| Vitamin B12 | 1.00 | mcg | Carbohydrates | 5.82 | g |
| Phosphorus | 1042.38 | mg | Protein | 0.15 | g |
| Potassium | 999.99 | mg | Fat | 0.06 | g |
| Calcium | 999.99 | mg | | | |

According to isotonicity calculations, the osmotic pressure of the preparation according to the invention is 630.1 kPa versus 799.5 kPa for a physiological saline solution vs. 763.0 kPa for blood at 37° C. The preparation according to the invention is, thus, slightly hypotonic.

Placebo: According to isotonicity calculations, the osmotic pressure of the placebo is 621.5 kPa versus 799.5 kPa for a physiological saline solution vs. 763.0 kPa for blood at 37° C. The placebo used is, thus, also slightly hypotonic. The placebo sample comprised a mixture of fructose, glucose, citric acid, orange flavour, carrot extract and aspartame as a sweetening agent.

TABLE 2
Form, Concentration and Composition of Micronutrients

| Nutrients per single dose | | | Composition per single dose | | |
|---|---|---|---|---|---|
| Vitamin B12 | 0.0003 | mg | Cyanocobalamine | 0.00033 | mg |
| Biotin | 0.0167 | mg | Biotin | 0.01667 | mg |
| Folic acid | 0.0667 | mg | Folic acid | 0.06667 | mg |
| Selenium | 0.0333 | mg | Sodium selenite | 0.11099 | mg |
| Molybdenum | 0.0667 | mg | Sodium molybdate | 0.16801 | mg |
| Chromium | 0.0667 | mg | Chromium-III-chloride | 0.341 | mg |
| | | | hexahydrate | | |
| Vitamin B2 | 0.53 | mg | Riboflavin | 0.530 | mg |
| Vitamin B1 | 0.47 | mg | Aneurin-HCl | 0.600 | mg |
| Vitamin B6 | 0.67 | mg | Pyridoxol-HCl | 0.790 | mg |
| Pantothenic acid | 2.00 | mg | Calcium pantothenate | 2.16 | mg |
| Vitamin E | 3.33 | mg | Dl-alpha-tocopherol | 3.33 | mg |
| Copper | 0.6700 | mg | Copper gluconate | 4.87 | mg |
| Niacin | 6.00 | mg | Nicotinic acid amide | 6.00 | mg |
| Manganese | 1.67 | mg | Manganese gluconate | 13.54 | mg |
| Zinc | 5.00 | mg | Zinc gluconate | 34.85 | mg |
| Iron | 5.00 | mg | Iron gluconate | 38.40 | mg |
| Beta-carotene | 0.54 | mg | beta-carotene 1% | 54.00 | mg |
| Vitamin C | 50.00 | mg | Sodium ascorbate | 56.00 | mg |

Accompanying measures: All of the 40 participants followed a one-hour workout programme three times a week under the supervision of a qualified trainer over the 10-week test period.

Examination parameters: Total cholesterol, HDL cholesterol, triglycerides, blood sugar, maximum performance (watts), maximum heart frequency (pulse/min), performance per kg/body weight, individualized anaerobic threshold performance (IAT in watts), individualized anaerobic threshold heart frequency (IATHF in pulse/min), individualized anaerobic threshold performance per kg body weight (IAT/kg, in watts). Performance-diagnostic parameters were determined on the ergometer. Threshold performances were determined according to the Conconi test (Boutellier, U.: Physiological basis for the measurement of aerobic capacity, Schweiz Rundsch. Med. Prax. (1989), 78(35): 921-4; Conconi F., et al.: Determination of the anaerobic threshold by a noninvasive field test in runners, J. Appl. Physiol. (1982), 52(4): 869-73).

Results: The trend study was completed by 29 persons. 11 participants had to be excluded because of lacking compliance.

The evaluation of the individual parameters revealed the following deviations in % after a 10-week examination period:

TABLE 3
Changes in Performance-Diagnostic Parameters

| | Changes in performance criteria in % | |
|---|---|---|
| | Verum | Placebo |
| Performance criterion | group (n = 12) | group (n = 17) |
| Maximum performance (watts) | 13.75 | 13.24 |
| Maximum heart frequency | −6.17 | −1.94 |
| (pulse/min) | | |
| Maximum performance/kg | 0.18 | 0.15 |
| KG(watts/kg) | | |
| IAT (watts) | 13.89 | 6.39 |
| IATHF (pulse/min) | −3.25 | 1.75 |
| IAT/kg (watts/kg) | 0.18 | 0.08 |

TABLE 4

Change in % (mg/100 ml)

| Laboratory | Verum | Placebo |
|---|---|---|
| value | group (n = 8) | group (n = 5) |
| Total cholesterol | −9.1% (from 221.4 | −8.15% (from 214.2 |
| | to 201.2) | to 196.8) |
| HDL cholesterol | +61.9% (from 43.3 | +45.5% (from 50.3 |
| | to 60) | to 73.4) |

Under identical training conditions, a marked increase in the individualized anaerobic threshold performance (IAT in watts) could be observed in the verum group. This corresponds to a shift to the right of the lactate curve. Alkaline salts (e.g., sodium bicarbonate; hydrogen carbonate) are involved in the regulation of the acid-base balance and serve, inter alia, to neutralize lactate formed during anaerobic energy supply. A high content of basic nutrients is able to increase the uptake of lactate from the muscle cell, i.e. minimize the pH drop in the muscle cell, thus counteracting symptoms of fatigue.

1-35. (Canceled)

36. A method of accelerating lactate degradation comprising:
obtaining a solution of salts of hydrogen carbonates or carbonates of B-complex vitamins; and
administering them to a subject in a manner effective to accelerate lactate degradation.

37. The method of claim 36, further defined as a method of improving endurance and efficiency in the subject.

38. The method of claim 36, further defined as a method of increasing the anaerobic threshold performance of the subject.

39. The method of claim 36, further defined as a method of preventing or slowing symptoms of fatigue in the subject.

40. The method of claim 36, wherein the solution is comprised in a sports drink.

41. The method of claim 36, wherein the solution further comprises one or more salts of organic acids having a C₁-C₆ base body.

42. The method of claim 41, wherein the one or more salts of organic acids having a C₁-C₆ base body is further defined as a citrate, hydrogen citrate, acetate, gluconate, tartrate, or salt of another C₂-C₆ acid.

43. The method of claim 36, wherein the salt is an Na, K, NH₄, Ca or Mg salt.

44. The method of claim 36, wherein the salt is an acetate or gluconate.

45. The method of claim 36, wherein the salt is a citrate or hydrogen citrate.

46. A liquid base-containing micronutrient mixture comprising an organic acid salt, further defined as a hydrogen carbonate or carbonate, B-complex vitamin, vitamin C, iron, chromium, selenium, zinc, manganese, and/or copper, wherein said micronutrient mixture has an osmotic pressure of 650 kPa or less in an aqueous solution.

47. The composition of claim 46, further defined as having a pH of 7.5 or more in an aqueous solution.

48. The composition of claim 47, further defined as having a pH of 8.0 or more in an aqueous solution.

49. The composition of claim 48, further defined as having a pH of 8.5 or more in an aqueous solution.

50. The composition of claim 46, further comprising vitamin E, provitamin A, molybdenum, magnesium, chloride, sodium, calcium, potassium, or phosphate.

51. The composition of claim 46, further defined as comprising carbohydrates in an amount of less than 30%, based on the total weight of the dry composition.

52. The composition of claim 51, further defined as comprising carbohydrates in an amount of less than 20%, based on the total weight of the dry composition.

53. The composition of claim 46, further defined as being substantially free of fats.

54. The composition of claim 46, further defined as being substantially free of proteins.

55. The composition of claim 46, further defined as comprising a B-complex vitamin further defined as vitamin B1, B2, B6, B12, biotin, folic acid, pantothenic acid, or niacin.

56. The composition of claim 46, further defined as comprising:
a hydrogen carbonate or carbonates, in an amount of from 0.2 to 20%;
a B-complex vitamin in amount of from 0.0001 to 2%;
vitamin C in an amount of from 0.001 to 5%;
iron in an amount of from 0.0001 to 0.5%;
chromium in an amount of from 0.000001 to 0.01%;
selenium in an amount of from 0.000001 to 0.01%;
zinc in an amount of from 0.0001 to 0.5%;
manganese in an amount of from 0.00001 to 0.1%; and
copper in an amount of from 0.00001 to 0.1%;
wherein each percentage is based on the total weight of the dry composition.

57. The composition of claim 46, further comprising a vegetable extract, fruit powder, organic acid, coloring agent, or sweetening agent.

58. The composition of claim 57, further defined as comprising citric acid.

59. The composition of claim 46, further defined as being in a dose form.

60. The composition of claim 59, further defined as being in a daily dose form or a single dose form.

61. The composition of claim 46, further defined as comprising cyanocobalamine, sodium selenite, sodium molybdate, chromium-III-chloride hexahydrate, riboflavin, aneurin-HCl, pyridoxine-HCl, calcium pantothenate, dl-alpha-tocopherol, copper gluconate, manganese gluconate, zinc gluconate, iron gluconate, or sodium ascorbate.

62. The composition of claim 46, further defined as comprising citric acid, orange fruit powder, carrot extract, calcium glycerophosphate, magnesium glycerophosphate, sodium chloride, or a sweetening agent.

63. The composition of claim 46, further defined as comprised in an aqueous solution at a concentration of from 0.5 to 200 g per 250 ml.

64. The composition of claim 63, further defined as comprised in an aqueous solution at a concentration of from 2 to 70 g per 250 ml.

65. The composition of claim 64, further defined as comprised in an aqueous solution at a concentration of from 5 to 20 g per 250 ml.

66. The composition of claim 46, further defined as comprising sodium, ammonium, potassium, or hydrogen carbonates.

67. The composition of claim 46, further defined as comprising selenium in selenite or selenate form.

68. The composition of claim 46, further defined as comprising metals in gluconate form.

69. The composition of claim 46, further defined as comprising sodium, potassium, calcium, magnesium carbonates or bicarbonates, sodium, potassium, calcium, magnesium salts of organic acids, mono- and hydrogen citrates, tartrate, gluconate and/or other organic salts.
